(19) United States
(12) Patent Application Publication
(10) Pub. No.: US 2009/0042987 A1
(43) Pub. Date: Feb. 12, 2009

(54) TREATMENT OF NEUROPATHIC PAIN

(76) Inventor: Michael Lionel Selley, Sydney (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/167,898

(22) Filed: Jul. 3, 2008

Related U.S. Application Data
(60) Provisional application No. 60/948,417, filed on Jul. 6, 2007.

(51) Int. Cl. A61K 31/196; C07C 229/42

(52) U.S. Cl. 514/563; 562/457

(57) ABSTRACT

A method of decreasing neuropathic pain in a mammal, comprising administering to said mammal an effective amount of tranilast for a period of time sufficient to decrease pain.

### FIELD OF THE INVENTION

The present invention relates generally to a method of treating neuropathic pain, including pain associated with diabetic retinopathy.

### BACKGROUND OF THE INVENTION

The physical causes of pain may be divided into two types: nociceptive and neuropathic pain. The differences are important for understanding the nature of the pain problem and especially for determining how to treat the pain.

Nociceptors are the nerves which sense and respond to parts of the body which suffer from damage. They signal tissue irritation, impending injury, or actual injury. When activated, they transmit pain signals. The pain is typically well localized, constant, and often with an aching or throbbing quality. Visceral pain is the subtype of nociceptive pain that involves the internal organs. It tends to be episodic and poorly localized. Examples include sprains, bone fractures, burns, bumps, bruises, inflammation (from an infection or arthritic disorder), obstructions, and myofascial pain (which may indicate abnormal muscle stresses). Nociceptive pain is usually time limited; resolving when tissue damage heals, although arthritis is an example of nociceptive pain that is chronic in nature. Nociceptive pain can usually be treated with NSAIDs, and with opoids.

Neuropathic pain is the result of an injury or malfunction in the peripheral or central nervous system. The pain can be triggered by an injury, but this injury may or may not involve actual damage to the nervous system. Nerves can be infiltrated or compressed by tumors, strangulated by scar tissue, or inflamed by infection. The pain frequently has burning, lancinating, or electric shock qualities. Persistent allodynia, pain resulting from a nonpainful stimulus such as a light touch, is also a common characteristic of neuropathic pain. The pain may persist for months or years beyond the apparent healing of any damaged tissues. In this setting, pain signals no longer represent an alarm about ongoing or impending injury, instead the alarm system itself is malfunctioning.

Examples of neuropathic pain include post herpetic (or post-shingles) neuralgia, reflex sympathetic dystrophy, components of cancer pain, phantom limb pain, entrapment neuropathy (e.g., carpal tunnel syndrome), and peripheral neuropathy (widespread nerve damage). Among the many causes of peripheral neuropathy, diabetes is the most common, but the condition can also be caused by chronic alcohol use, exposure to other toxins (including many chemotherapeutic agents), vitamin deficiencies, and in many cases the cause is idiopathic.

Neuropathic pain can be very difficult to treat. Sometimes strong opioid analgesics may provide only partial relief. Several classes of medications not normally thought of as analgesics may be effective, alone or in combination with opioids and other treatments. These include tricyclic antidepressants such as amitriptyline, and anticonvulsants such as gabapentin and pregabalin.

Unfortunately, neuropathic pain often responds poorly to standard pain treatments and occasionally may get worse instead of better over time. For some people, it can lead to serious disability. Methods of improved treatment for neuropathic pain are of great interest. The present invention addresses this need.

### SUMMARY OF THE INVENTION

Methods are provided for the treatment of neuropathic pain in an individual, by administering an effective dose of a compound of formula (I). In some embodiments the compound is orally administered. A particularly preferred compound of formula (II) for use in the invention is 2-[[3-(3,4-dimethoxyphenyl)-1-oxo-2-propenyl]amino]benzoic acid (tranilast, TNL). In some embodiments of the invention the neuropathic pain is a result of diabetic neuropathy.

In some embodiments of the invention, the compound is administered at a dose effective in decreasing neuropathic pain every other day, daily, twice daily, etc., for a period of at least one day, at least two days, at least three days, at least one week, or longer to achieve a decrease in pain.

One aspect of the present invention is directed to a method for inducing analgesia of neuropathic pain in a subject, the method comprising administering to said subject an effective amount of a compound of formula (I). In another aspect there is provided a method for prophylactically inducing analgesia from neuropathic pain in a subject, said method comprising administering to said subject an effective amount of a compound of formula (I)

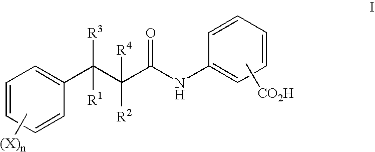

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

The carboxyl group may be in the 2-, 3- or 4-position of the aromatic ring. Preferably the carboxyl group is in the 2-position.

Preferably at least one of R¹ and R² is a hydrogen atom. More preferably, both of R¹ and R² are hydrogen atoms.

Preferably R³ and R⁴ taken together form a chemical bond. Such compounds having an unsaturated bond may be in the form of E or Z geometric isomers.

Preferably n is 1 or 2 and each X, which may be the same or different, is selected from halogen, C₁-C₄ alkyl or C₁-C₄alkoxy. Preferably X is selected from halogen and C₁-C₄alkoxy. More preferably, n is 2 and both X are selected from C₁-C₄alkoxy, especially when both X are methoxy.

In one preferred embodiment the compound is 3-hydroxykynurenic acid (3-HKA), 3-hydroxyanthranilic acid (3-HAA), picolinic acid (PA) or quinolinic acid (QA).

One aspect of the present invention provides a method for inducing analgesia for neuropathic pain in a subject, said method comprising administering to said subject an effective amount of tranilast.

Yet another aspect of the present invention is directed to the use of a compound of formula I thereof in the manufacture of a medicament for the treatment of neuropathic pain.

### BRIEF DESCRIPTION OF THE DRAWINGS

FIG. 1 is a graph depicting mean±SE water intake of rats at day 7-10 post STZ administration.

FIG. 2 is a graph depicting mean±SE blood glucose levels in rats post STZ administration.

FIG. 3 is a graph depicting mean±SE body weight (g) of rats following STZ administration.

FIG. 4 is a graph depicting mean±SE PWT values for the left and right hindpaws in adult male SD rats prior to and at, 2, 4, and 8-11 weeks post-STZ injection.

FIG. 5 is a graph depicting mean±SE PWT versus tome curvies following administration of the 7^{th} consecutive oral bolus dose of vehicle or tranilast at 10, 30, 100, 200 or 300 mg/kg c.f. the 7^{th} consecutive s.c. bolus dose of gabapentin at 100 mg/kg. Each STZ-diabetic rat received multiple dosing regimens comprising twice-daily dosing for 7 consecutive doses, according to a washout protocol.

FIG. 6 is a graph depicting baseline PWT values assessed prior to oral administration of doses 1, 3, 5 and 7 for tranilast at 100, 200, 300 or 400 mg/kg, compared with s.c. gabapentin at 10 mg/kg or vehicle, administered according to a twice-daily dosing regimen for 7 consecutive doses in drug naïve STZ-diabetic rats.

FIG. 7 is a graph depicting mean±SE PWT versus time curves following administration of the 7^{th} consecutive oral bolus dose of tranilast at 100, 200, 300 or 400 mg/kg administered according to a twice-daily dosing regimen, compared with the corresponding response produced by the 7^{th} consecutive bolus dose of gabapentin at 100 mg/kg or vehicle, administered according to a twice-daily dosing regimen in drug naïve STZ diabetic rats.

FIG. 8 is a graph depicting mean±SE % maximum possible reversal of mechanical allodynia at the time of peak response (AUC) following administration of the 7^{th} consecutive oral bolus dose of tranilast at 100, 200, 300 or 400 mg/kg administered according to a twice-daily dosing regimen, compared with the corresponding response produced by the 7^{th} consecutive bolus dose of gabapentin at 100 mg/kg or vehicle, administered according to a twice-daily dosing regimen in drug naïve STZ diabetic rats. * significantly different from vehicle (p<0.05).

FIG. 9 is a graph depicting mean±SE area under the PWT versus time curve (AUC) following administration of the 7^{th} consecutive oral bolus dose of tranilast at 100, 200, 300 or 400 mg/kg administered according to a twice-daily dosing regimen, compared with the corresponding response produced by the 7^{th} consecutive bolus dose of gabapentin at 100 mg/kg or vehicle, administered according to a twice-daily dosing regimen in drug naïve STZ diabetic rats. * significantly different from vehicle (p<0.05).

FIG. 10 is a flow chart providing considerations for therapy of neuropathic pain.

### DETAILED DESCRIPTION OF THE INVENTION

The present invention relates to the treatment of neuropathic pain with a compound of formula I, particularly treatment with orally administered tranilast. The methods of the invention reduce neuropathic pain, e.g. as measured by a decrease in tactile allodynia when quantitated in an experimental animal model. The effect is dose-dependent, where the effective dose is the dose that is sufficient to decrease pain, e.g. from around about 50 to 5000 mg/kg. of tranilast.

The existing treatments for neuropathic pain are not satisfactory in that many patients experience dose-limiting side-effects with these medications, and approximately 50% of patients do not achieve adequate pain relief with any one treatment. This large unmet medical need emphasises the need for novel therapeutics for the alleviation of neuropathic pain.

Tranilast (N-(3′,4′-dimethoxycinnamonyl)anthranilic acid), has been shown to have a variety of clinical effects, including anti-inflammatory effects. The tranilast-mediated effects in restenosis are attributed to the suppression of transforming growth factor-β₁ (TGF-β₁) synthesis and interference with growth factor-mediated proliferation and migration of fibroblasts and vascular smooth muscle cells.

It should be understood that the neural cell treated in accordance with the method of the present invention is may be located in a mammal, therefore requiring the subject method to be performed in vivo. Where the subject cell is one of a group of cells or a tissue, either isolated or not, the subject method may modulate the functioning of all the cells in that group or just a subgroup of cells in that group. Similarly, in the context of the modulation of the biological functioning of a mammal, it should be understood that the subject modulation may be achieved in the context of modulating cell functioning either systematically or in a localized manner. Still further, irrespective of which means is employed, the cellular impact of the change in cell functioning may occur in the context of either all cells or just a subgroup of cells within the relevant environment.

Reference to decreasing neuropathic pain should be understood as a reference to preventing, reducing or otherwise inhibiting one or more aspects of said activity.

The term “mammal” as used herein includes humans; primates; livestock animals, e.g. sheep, pigs, cattle, horses, donkeys, etc.; laboratory test animals e.g. mice, rabbits, rats, guinea pigs; companion animals e.g. dogs, cats; and captive wild animals e.g. foxes, kangaroos, deer; and the like. Preferably, the mammal is human or a laboratory test animal. Even more preferably, the mammal is a human.

### Neuropathic Pain

Neuropathic pain refers to pain that originates from pathology of the nervous system. Diabetes, infection (herpes zoster), nerve compression, nerve trauma, “channelopathies,” and autoimmune disease are examples of diseases that may cause neuropathic pain. Neuropathic pain reflects both peripheral and central sensitization mechanisms. Abnormal signals arise not only from injured axons but also from the intact nociceptors that share the innervation territory of the injured nerve. Neuropathic pain may result from lesions of the central nervous system, or from the peripheral nervous system.

Neuropathic pain is distinguished from other pain conditions where the pain generator begins with disease of normeural tissues. These normeuropathic pain entities are said to be nociceptive and include conditions such as osteoarthitis and inflammatory pain. By definition, neuropathic pain originates from a lesion of the nervous system (central and/or peripheral). Innumerable diseases or conditions may be the culprits. Examples include but are not limited to autoimmune disease, e.g. multiple sclerosis, metabolic diseases e.g. diabetic neuropathy (including peripheral, focal, proximal and autonomic), infection e.g. shingles, postherpetic neuralgia, vascular disease, trauma, pain resulting from chemotherapy, HIV infection/AIDS, spine or back surgery, post-amputation pain, central pain syndrome, postherpetic neuralgia, phantom limb, trigeminal neuralgia, reflex sympathetic dystrophy syndrome, nerve compression, stroke, spinal cord injury and cancer. Generally the lesion leading to pain can directly involve the nociceptive pathways.

The importance of primary afferent inputs in neuropathic pain is strongly suggested by several pharmacological studies. Ongoing peripheral neuronal input is critically involved in the maintenance of neuropathic pain. Although the data suggests that an injured afferent nerve is responsible for neuropathic pain, hyperalgesia can also develop in the absence of neural activity from the injured nerve. In other cases, the intact nociceptors that survive injury and that innervate the region affected by the transected nerve fibers sensitize and have spontaneous activity. These changes in the intact nociceptors may induce ongoing pain and may account for certain aspects of hyperalgesia, e.g. in sympathetically maintained pain.

A variety of voltage-gated sodium channels are expressed in the primary afferent neurons that are critical for the initiation and generation of action potentials in the neuronal membranes. The accumulation and increased membrane density of Na⁺ channels following axotomy causes the ectopic discharges of neuropathic pain. At least six subtypes of sodium channels are reported to be present in the dorsal route ganglia neurons which are subdivided into major categories depending on their sensitivity to the neurotoxin tetrodotoxin (TTX). The TTX-sensitive Na⁺ channels are mainly expressed in the myelinated A fibres, while the TX-resistant Na⁺ channels are predominantly expressed on the unmyelinated nociceptive C-firbres. Although downregulation of TTX-resistant Na⁺ channels has been reported following axotomy recent findings suggest their redistribution in uninjured nerve fibres for the development of neuropathic pain.

The primary sensory neurons express a number of peptides that act as neurotransmitters and neuromodulators. After peripheral axotomy, neuropeptides such as substance P and calcitonin gene-related peptides and somatostatin, which are abundantly present in sensory neurons, are down-regulated, while neuropeptides such as vaso-active intestinal peptide, galanin, neuropeptide Y (NPY) and cholecystokinin (CCK), which are normally expressed at low levels in sensory neurons, are dramatically increased. The neuropeptides in sensory neurons have a distinctive role in mediating neuropathic hyperalgesia. In the SNL model of neuropathic pain, the NPY expression was increased in the medium and large diameter DRG neurons, spinal dorsal horn and nucleus gracilis and microinjection of NPY antiserum or NPY receptor antagonist to the nucleus attenuated the tactile hyperalgesia. The upregulation of CCK in primary sensory neurons is reported to be involved in the insensitivity of morphine in neuropathic pain.

Peripheral nerve injury triggers sprouting of noradrenergic sympathetic axons into the sensory dorsal root ganglia, providing evidence of a sympathetic component in neuropathic pain. In most of the experimental animal models of neuropathic pain, a major component of the neuropathic pain symptoms have been relieved by sympathectomy. Sympathetic sprouting also contributes to the ectopic and spontaneous discharge of the injured nerve fibres. The neurotrophin nerve growth factor and brain-derived neurotrophic factor have been implicated in the mechanisms of sympathetic sprouting to the dorsal root ganglion neurons following nerve injury.

Peripheral nerve injury is associated with a local inflammatory reaction of the nerve trunk and inflammatory mediators sensitize the axotomised nerve fibers. Thus proinflammaory mediators may be involved in the development and maintenance of neuropathic hyperalgesia. Bradykinin is released as a result of tissue damage which has been mainly associated with inflammatory hyperalgesia. However, it has been shown that antagonists of bradykinin receptors have antihyperalgesic effects. The prostaglandins, including PGE₂ and PGI₂, are also rapidly produced following tissue injury and are major contributors to inflammatory pain. It has been reported that cyclooxygenase inhibitors, which inhibit the production of prostaglandins, attenuate the thermal and mechanical hyperalgesia in animal models of neuropathic pain.

It is generally accepted that the medium diameter thinly myelinated A- and small-diameter unmyelinated C-fibres terminate in dorsal horn laminae I and II respectively, while the large-diameter myelinated Aβ fibres terminate in laminae III and IV. It has been postulated that peripheral nerve injury results in withdrawal of C-fibre central axon terminals from the outer lamina II and the terminals of large myelinated Aβ fibres sprout to the site of C-fibre terminals to innervate the various synaptic sites. The new synaptic connections release excitatory transmitters such as amino acids and peptides that are not normally found in synapses related to these connections. Aβ fibres are associated with cutaneous mechanoreceptors propagating the sense of touch. They have no modulatory effect on pain sensation. With the development of abnormal connections to lamina II, however, light touch becomes transmissible as pain. This phenomenon appears, at least in part, to explain the symptom of mechanical allodynia in which a normal innocuous cutaneous sensation is perceived as severe pain. This symptom is often particularly distressing for diabetics because one of the first areas to be affected is the soles of the feet. This may be because the long sensory axons are particularly susceptible to metabolic insult. Diabetics with neuropathic pain in the feet often describe a sensation similar to walking on pebbles or broken glass which can greatly restrict mobility.

Peripheral nerve injury also causes the sensitisation of spinal dorsal horn neurons and subsequent facilitation of spinal excitability. Central sensitization is characterised by the presence of wind-up or long-term potentiation (LTP) where a short-lived volley of nociceptive stimulation results in the increase of post-synaptic potentials for a longer time. It has been shown that glutamate N-methyl-D-aspartate (NMDA) receptors play a role in the process of central sensitization.

There is evidence of a significant contribution of supraspinal influences in the development and maintenance of neurotrophic hyperalgesia. The supraspinal contribution of neuropathic pain was confirmed by the prevention of neuropathic hyperalgesia by the spinal transaction or inactivation of supraspinal sites in animal models. The spinal pain transmission system is under both inhibitory and excitatory control from the supraspinal sites, particularly the brainstem rostral ventromedial medulla (RVM). The degree of descending inhibitory control is substantially reduced in neuropathic animals. On the other hand, the descending facilitation effect from the RVM on spinal dorsal horn neuronal transmission is increased following peripheral nerve injury.

Spinal cord glial cells, particularly astrocytes and microglia, are activated by neuronal activation in the periphery. Substances, such as calcitonin gene-related peptide (CGRP), substance P and glutamate, released from the presynaptic terminals of the neurons which carry the message of the peripheral injury, activate glial cells and make them produce pro-inflammatory cytokines which may further increase the neuronal excitability. Both astrocytes and micoglia can release pro-inflammatory cytokines on activation and glia and neurons express receptors for them. The peri-spinal injection of antagonists of pro-inflammatory cytokine function prevents and/or reverses allodynia and hyperalgesia in several animal models. The fact that established allodynia and hyperalgesia can be reversed by pro-inflammatory cytokine antagonists supports the conclusion that these glial proteins are involved in the maintenance, as well as the initial induction, of these enhanced nociceptive states. It has been shown in a rat model of neuropathic pain induced by sciatic nerve inflammation (sciatic inflammatory neuropathy, SIN) that minocycline, a selective inhibitor of microglial cell activation, inhibited low threshold mechanical allodynia. Minocycline was able to attenuate established SIN-induced allodynia one day but not one week later. The data were consistent with a crucial role for microglial cells in initiating, rather than maintaining enhanced pain responses. Quantitative real-time RT-PCR measurement of spinal microglial and astrocytic activation markers in a rat model of neuropathic pain demonstrated that peripheral nerve injury induces an early spinal microglial activation that precedes astrocytic activation using mRNA for cell marker expression. The delayed but sustained expression of mRNA coding for glial fibrillary acidic protein (GFAP) was considered to implicate astrocytes in the maintenance phase of persistent pain states. It has been postulated that glial activation could be the driving force, maintaining the pain sensation even after the original injury has healed.

Specific types of neuropathic pain treatable with the methods of the invention include Tic douloureux. Without treatment, this is a debilitating disorder that involves attacks of severe pain in the facial area (also referred to as trigeminal neuralgia). Often there is little or no pain between attacks. The lightening-like attacks are referred to one of the dermatomes (V1, V2, or V3). Light touching of the skin in a so-called trigger zone suffices to evoke an attack. The disease appears to be associated with mechanical distortion at the entry zone of the nerve root to the brainstem. Demyelination may be seen at the compression site. Nerve compression from an aberrant blood vessel is one of the more common causes.

Another example of neuropathic pain condition is diabetic neuropathy. Diabetes often causes a length-dependent neuropathy (meaning that the longest axons in the peripheral nerve are most vulnerable). Patients report bilateral burning pain in the toes and feet. Quantitative sensory testing reveals decreased pain sensibility (with or without decreased touch sensibility).

Polyneuropathy affects approximately 30-50% of all diabetic patients and is the most common form of neuropathy. Diabetic polyneuropathy encompasses several neuropathic syndromes but the most common is distal symmetrical sensory polyneuropathy (DSP). The main clinical features of DSP are foot ulceration that can lead to amputation and painful diabetic neuropathy (PDN) leading to high rates of patient morbidity and mortality. The major determinants of DSP are glycaemic control and duration of diabetes. Macrovasular disease such as hypertension, hyperlipidaemia and smoking are also independent risk factors.

The prevalence rate of PDN is 7-20%, the variation reflecting the different criteria used to define neuropathic pain. It was found that nearly 25% of type 1 diabetic patients in the European Diabetes (EURODIAB) prospective study developed neuropathic symptoms over a 7-year period. It can be concluded that a high proportion of diabetic patients suffer from neuropathic pain.

The pain of PDN has been described in various terms as burning, “pins and needles”, lancinating, shooting like an electric shock, cramping, aching, contact hypersensitivity (allodynia) and numbness in the legs. Some patients experience walking as being barefoot on pebbles or scalding sand. There may be only mild symptoms in the toes while other patients may have continuous pain involving both legs and extending to the upper limbs.

The PDN associated with DSP is associated with a rapid increase of unpleasant sensory symptoms within weeks. This leads to persistent burning pain in the lower limbs, paraesthesiae and allodynia with a nocturnal exacerbation of symptoms. Depression and precipitous weight loss may also occur. Sensory loss is often mild or absent and there are no motor signs. Neuropathic pain may also present acutely in the context of poor glycaemic control, typically in type 1 subjects.

Postherpetic neuralgia is a complication of shingles and is an example of how an infection can lead to pain. Shingles results from an activation of the herpes zoster virus that takes up residence in the dorsal root ganglion after a chickenpox infection. The shingles eruption consists of blisters that follow the dermatome(s) of one or more spinal nerves. The blisters heal in time, but the pain may continue. Allodynia is a particularly prominent feature of postherpetic neuralgia. This allodynia may be present even with loss of C-fiber innervation of the epidermis.

Other conditions of interest for treatment include peripheral neuropathy, neuropathic pain associated with multiple sclerosis, phantom limb pain, pain from certain cancers, and the like.

Decrease in pain in humans can be monitored by the subject's evaluation, by sensitivity to touch, etc. Animal models also find use in quantitation of pain. Spontaneous foot lifting or paw withdrawal in animal models may provide a behavioral measure of pain. Alternative measures of ongoing pain involve the use of cellular markers of increased neuronal activity. Increased expression of the immediate early gene protein, c-Fos, in the dorsal horn (and perhaps other more rostral sites) is an example. Small-animal functional magnetic resonance imaging (fMRI) and/or PET imaging offer alternative measures. An example of a paw-withdrawal method in rodents with the SNL model is described by LaBuda and Little (2005) J Neurosci Methods. 144:175-181, herein specifically incorporated by reference for teachings of pain analysis methods.

### Compounds for Treatment of Neuropathic Pain

Compounds for use in the methods of the invention may have the structure of formula I

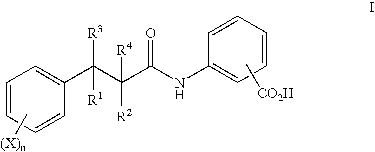

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3.

The carboxyl group may be in the 2-, 3- or 4-position of the aromatic ring. Preferably the carboxyl group is in the 2-position.

Preferably at least one of R¹ and R² is a hydrogen atom. More preferably, both of R¹ and R² are hydrogen atoms.

Preferably R³ and R⁴ taken together form a chemical bond. Such compounds having an unsaturated bond may be in the form of E or Z geometric isomers.

Preferably n is 1 or 2 and each X, which may be the same or different, is selected from halogen, C₁-C₄ alkyl or C₁-C₄alkoxy. Preferably X is selected from halogen and C₁-C₄alkoxy. More preferably, n is 2 and both X are selected from C₁-C₄alkoxy, especially when both X are methoxy.

A particularly preferred compound of formula (II) for use in the invention is 2-[[3-(3,4-dimethoxyphenyl)-1-oxo-2-propenyl]amino]benzoic acid (tranilast, TNL). In other embodiments the compound is 3-hydroxykynurenic acid (3-HKA), 3-hydroxyanthranilic acid (3-HAA), picolinic acid (PA) or quinolinic acid (QA).

Particularly preferred compounds useful in the invention are those of formula (II):

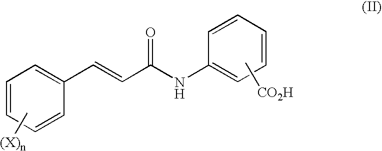

Examples of compounds of formula (II) include
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

A particularly preferred compound of formula (II) for use in the invention is 2-[[3-(3,4-dimethoxyphenyl)-1-oxo-2-propenyl]amino]benzoic acid (tranilast, TN L).

As used herein, the term “C₁-C₄alkyl” refers to linear or branched hydrocarbon chains having 1 to 4 carbon atoms. Examples of such groups include methyl, ethyl, n-propyl, isopropyl, n-butyl, sec-butyl and tert-butyl.

As used herein, the term “C₂-C₄alkenyl” refers to linear or branched hydrocarbon chains having 2 to 4 carbon atoms and one or two double bonds. Examples of such groups include vinyl, propenyl, butenyl and butadienyl.

As used herein, the term “C₁-C₄alkoxy” refers to hydroxy groups substituted with linear or branched alkyl groups having 1 to 4 carbon atoms. Examples of such groups include methoxy, ethoxy, n-propoxy, isopropoxy, n-butoxy, sec-butoxy and tert-butoxy.

As used herein, the term “halogen” or “halo” refers to fluoro, chloro or bromo atoms.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quaternised with such agents as lower alkyl halide, such as methyl, ethyl, propyl, and butyl chlorides, bromides and iodides; dialkyl sulfates like dimethyl and diethyl sulfate; and others.

Compounds of formula (I) and their pharmaceutically acceptable salts are known and may be prepared by methods known in the art, see U.S. Pat. No. 3,940,422 the contents of which are incorporated herein by reference.

It will also be recognised that some compounds of formula (I) may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

Without limiting the present invention to any one theory or mode of action, the compounds of formula (I) are orally active anti-allergic compounds. A particularly preferred compound of the invention is known by either of the chemical names N-[3,4-dimethoxycinnamoyl]-anthranilic acid or 2-[[3-(3,4-dimethoxyphenyl)-1-oxo-2-propenyl]amino]benzoic acid and may also be referred to as Tranilast. Still further, it is known by the chemical formula C₁₈H₁₇NO₅ and by the trade name Rizaben. The structure of N-[3,4-dimethoxycinnamoyl]-anthranilic acid is depicted below:

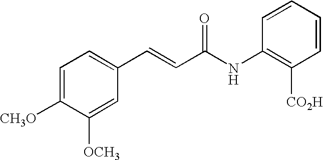

The metabolites, derivatives and compounds of formula (I), formula (II) or pharmaceutically acceptable salts thereof may also be used in conjunction with another therapy, for example an analgesic treatment regime. In some embodiments, the use of Tranilast or other compounds of the invention allow the use of a lower dose of a second drug than would ordinarily be used.

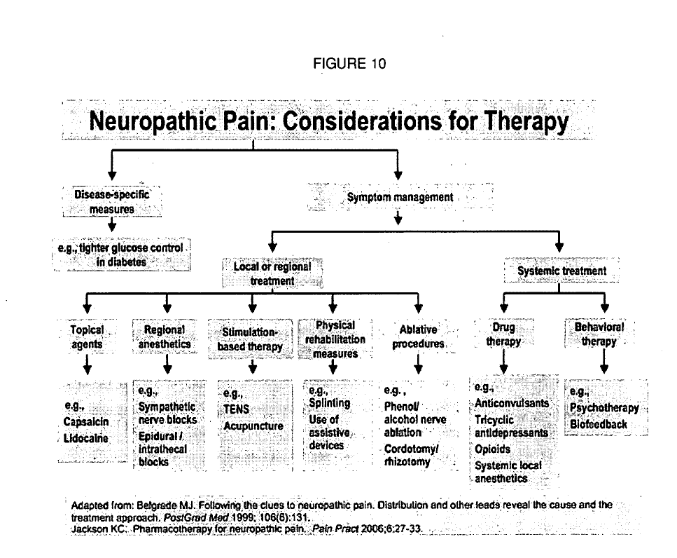

In some aspects of the invention, compounds of the invention are combined with one or more Standards of Care for treating neuropathic pain. Standards of Care for treating neuropathic pain are known, and include monotherapy, adjunct therapy and polytherapy. Examples of Standards of Care for neuropathic pain are disclosed in Clinical Journal of Pain; Volume 5(3), April 2004, Clinical Characteristics and Economic Costs of Patients with Painful Neuropathic Disorders. Integrative or combination treatment options are also provided in FIG. 10. For example, in some embodiments, compounds of the invention are administered in conjunction with topical agents, regional anesthetics, stimulation based therapy, physical rehabilitation measures, ablative procedures, drug therapy, behavioral therapy or a combination thereof.

Drugs useful in combination therapy with the compounds of the present invention include tricyclic antidepressants, whose analgesic actions may be attributable to noradrenaline and serotonin reuptake blockade, presumably enhancing descending inhibition, NMDA receptor antagonism and sodium-channel blockade. The mixed serotonin-noradrenaline reuptake inhibitor duloxetine has been used in the treatment of PDN. Carbamazipine has been used for the treatment of PDN but it has significant adverse effects making it a poor candidate for first line therapy. Gabapentin, an α-2 delta subunit voltage-gated calcium channel antagonist, has demonstrated efficacy against PDN. Pregabalin is a gabapentin analogue with a similar mechanism, higher calcium-channel affinity and better bioavailability. There is evidence of the effectiveness of opioid analgesics in PDN. Tramadol is a weak opioid and a mixed serotonin-noradrenaline reuptake inhibitor that is effective in the treatment of PDN. The lidocaine patch 5% has been shown to reduce the intensity of pain in PDN. Capsaicin, an ingredient of hot peppers is a substance P antagonist which has shown some efficacy in PDN.

Examples of therapeutic agents useful in combination therapy with compounds of the invention include, but are not limited to Darvocet N 50 mg propoxyphene+325 mg APAP (APAP=acetaminophen); Darvocet N 100 100 mg propoxyphene+650 mg APAP; Percocet 2.5 mg oxycodone+325 mg APAP or 5 mg oxycodone+325 mg APAP or 7.5 mg oxycodone+325 mg APAP or 7.5 mg oxycodone+500 mg APAP or 10 mg oxycodone+650 mg APAP; Percodan, Endodan 5 mg oxycodone+325 mg aspirin; Roxicet, Endocet 5 mg oxycodone+325 mg APAP; Roxilox, Tylox 5 mg oxycodone+500 mg APAP; Lorcet-HD 5 mg hydrocodone+500 mg APAP; Lorcet Plus 7.5 mg hydrocodone+650 mg APAP; Lorcet 10/650 10 mg hydrocodone+650 mg APAP; Lortab 2.5/500 2.5 mg hydrocodone+500 mg APAP; Lortab 5/500; Vicodin 5 mg hydrocodone+500 mg APAP; Lortab 7.5/500; Vicodin ES 7.5 mg hydrocodone+500 mg APAP; Lortab 10/500 10 mg hydrocodone+500 mg APAP; Vicoprofin 7.5 mg hydrocodone+200 mg ibuprofen; Tylenol #3 30 mg codeine+300 mg APAP; Ultracet 37.5 mg tramadol+325 mg APAP; benzodiazepines (e.g., lorazepam, clonazepam); neuroleptics (e.g., haloperidol); corticosteroids (e.g., dexamethasone, prednisone); stimulants (e.g., methylphenidate) or a combination thereof.

Additional examples of therapeutic agents include one or more opioid agents useful in the compositions and methods of the invention, which include but are not limited to morphine, codeine and thebaine, hydromorphone, hydrocodone, oxycodone, oxymorphone, desomorphine, diacetylmorphine (Heroin), nicomorphine, dipropanoylmorphine, benzylmorphine and ethylmorphine; s fentanyl, pethidine, methadone, and propoxyphene; endorphins, enkephalins, dynorphins, and endomorphins.

Examples of non-opioid analgesic agents useful in the compositions and methods of the invention include but are not limited to acetaminophen; a non-steroidal anti-inflammatory drug (NSAID) such as a salicylate (including, for example, amoxiprin, benorilate, choline magnesium salicylate, diflunisal, faislamine, methyl salicylate, magnesium salicylate), an arylalkanoic acid (including, for example, diclofenac, aceclofenac, acemetacin, bromfenac, etodolac, indometacin, nabumetone, sulindac, tolmetin), a profen (including, for example, ibuprofen, carprofen, fenbuprofen, flubiprofen, ketaprofen, ketorolac, loxoprofen, naproxen, suprofen), a fenamic acid (including, for example mefenamic acid, meclofenamic acid), an oxicam (including, for example, piroxicam, lomoxicam, meloxicam, tenoxicam), a pyrazolidine derivative (including, for example, phenylbutazone, azapropazone, metamizole, oxyphenbutazone, sulfinprazone); a Cox-2 inhibitor (such as valdecoxib, celecoxib, or rofecoxib), a local analgesic (such as lidocaine or mexiletine); an anti-depressant (such as amitriptyline, carbamazepine, gabapentin, or pregabalin) an atypical analgesic (such as orphenadrine, cyclobenzaprine, scopolamine, atropine, or gabapentin), a psychotropic agent (such as tetrahydrocannabinol), an NMDA receptor antagonist (such as ketamine), an α2-adrenoreceptor agonists (such as clonidine) and a synthetic drug having narcotic properties such as tramadol. In one embodiment the non-opioid analgesic agent is acetaminophen, naproxen.

Stimulant agents useful in the methods and compositions of the invention include, but are not limited to, aminophylline, caffeine, dyphlline, oxitriphylline, theophhylline, amphetamine, benzphetamine, dextroamphetamine, diethylpropion, mazindol, methamphetamine, methylphenidate, dexmethylphenidate, pemoline, sibutramine, modafinil, atomoxetine, phendimetrizine, phenteramine, adrafinil, phenylpropanolamine, psuedoephedrine, synephrine, amphetaminil, furfenorex, or a combination thereof.

Barbiturate agents useful in the methods and compositions of the invention include, but are not limited to, Allobarbital Alphenal, Amobarbital, Aprobarbital, Barbexaclone, Barbital, Brallobarbital, Butabarbital, Butalbital, Butobarbital, Butallylonal, Crotylbarbital, Cyclobarbital, Cyclopal, Ethallobarbital, Febarbamate, Heptabarbital, Hexethal, Hexobarbital, Mephobarbital, Metharbital, Methohexital, Methylphenobarbital, Narcobarbital, Nealbarbital, Pentobarbital, Primidone, Probarbital, Propallylonal, Proxibarbal, Proxibarbital, Reposal, Secbutabarbital, Secobarbital, Sigmodal, Talbutal, Thialbarbital, Thiamylal, Thiobarbital, Thiobutabarbital, Thiopental, Valofane, Vinbarbital, Vinylbital, 1,3-dimethoxymethyl 5,5-diphenyl-barbituric acid (DMMDPB), 1-monomethoxymethyl 5,5-diphenylbarbituric acid (MMMDPB) an diphenyl-barbituric acid (DPB) and their precursors, derivatives and analogs or a combination thereof.

Compounds and methods of the invention can be incorporated into dosage forms, such as those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,556; 5,871,776; 5,902,632; and 5,837,284. In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administrations of the two types of molecules. These molecules may be administered in any order.

An “effective” amount means an amount necessary at least partly to attain the desired response, or to delay the onset or inhibit progression or halt altogether, neuropathic pain. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Administration of the compounds of formula (I), formula (II) or pharmaceutically acceptable salts thereof, in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable, for example, from about 0.5 mg/kg, 5 mg/kg, about 10 mg/kg, about 100 mg/kg, about 500 mg/kg, about 1000 mg/kg may be administered per kilogram of body weight per day. Dosage regimes may be adjusted to provide the optimum therapeutic response. For example, several divided doses may be administered thrice daily, twice daily, daily, weekly, monthly or other suitable time intervals or the dose may be proportionally reduced as indicated by the exigencies of the situation. Usually the regimen will be maintained for at least about 2 days, at least about 3 days, at least about 5 days, at least about 1 week, or longer.

The modulatory agent may be administered in a convenient manner such as by the oral, intravenous (where water soluble), intraperitoneal, intramuscular, subcutaneous, intradermal or suppository routes or implanting (eg. using slow release molecules). The modulatory agent may be administered in the form of pharmaceutically acceptable nontoxic salts, such as acid addition salts or metal complexes, eg. with zinc, iron or the like (which are considered as salts for purposes of this application). Illustrative of such acid addition salts are hydrochloride, hydrobromide, sulphate, phosphate, maleate, acetate, citrate, benzoate, succinate, maleate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

The agent may be linked, bound or otherwise associated with any proteinaceous or non-proteinaceous molecules. For example, in one embodiment of the present invention said agent may be associated with a molecule which permits targeting to a localised region.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant, preferably oral.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg of active compound, about 100 μg, about 1 mg, about 10 mg, about 25 mg, about 100 mg, about 200 mg/kg, and not more than about 2000 mg.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Yet another aspect of the present invention relates to the metabolites or derivatives as hereinbefore defined or pharmaceutically acceptable salts thereof or antagonists thereof, as hereinbefore defined, when used in the method of the present invention.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference in their entirety as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims. The present invention is further defined by the following non-limiting examples:

### EXPERIMENTAL

### Methods

Experimental Animals Ethical approval for this study was obtained from the Animal Experimentation Ethics Committee of The University of Queensland. Adult male Sprague-Dawley (SD) rats (222±4 g) were used for this study. Rats were housed in a temperature controlled room (21+2.0 C) with a 12/12 hour light/dark cycle. Food and water were available ad libitum. Initiation of drug administration occurred at 8-11 weeks post-STZ administration for rats in Experiment 1 (PCT-001) and at 10-12 weeks post-STZ administration for rats in Experiment 2 (PCT-007).

### Materials and Chemicals

Isoflurane (Forthane) was obtained from Abbott Australasia Pty Ltd (Sydney, Australia) and sodium benzylpenicillin vials were purchased from CSL Ltd (Melbourne, Australia). Ketamine and xylazine injection vials were purchased from Provet (Brisbane, Australia). Streptozotocin (STZ), citric acid and trisodium citrate were purchased from the Sigma Chemical Company (Sydney, Australia). Saline ampoules were obtained from Astra Pharmaceuticals Pty Ltd (Sydney, Australia). Medical grade O₂ and CO₂ were purchased from BOC Gases Australia Ltd (Brisbane, Australia). Polyethylene tubing (O.D. 0.8 mm×I.D. 0.5 mm) was purchased form Dural Plastics and Engineering Pty. Ltd. (Sydney, Australia) and silk sutures (Dysilk Black Braided Siliconised Silk) were obtained from Dynek Pty Ltd (Hendon, Australia).

### Compounds for Administration

Test Article Tranilast was supplied by the Sponsor and stored at −4° C. until use. Gabapentin (100 mg/kg, dissolved in water for injection) was utilized as the positive control for this study.

Test Article and Vehicle Preparation Test article formulations were prepared freshly on the day of administration by weighing the appropriate amount of bulk powder and suspending it in the required volume of vehicle. Carboxymethyl cellulose ((CMC) 0.5% in water for injection) was utilized as the vehicle for this study.

Test Article Administration. Rats were lightly anaesthetized with 50% O₂:50% CO₂ to facilitate the administration of oral bolus doses of each of the test article (tranilast), gabapentin or vehicle. The maximum oral gavage volume administered in this study was 500 L. 4.4

### Induction of Painful Diabetic Neuropathy

STZ-Diabetic Rat Model of Painful Diabetic Neuropathy. Painful diabetic neuropathy is a common long-term complication of diabetes in humans that develops as a result of a sustained biochemical nerve injury. The most commonly used rat model of this condition involves the administration of the chemical, streptozotocin (STZ) to rats, resulting in destruction of the -cells in the pancreas, thereby rendering the rats diabetic due to the markedly reduced insulin-secreting capacity. Following the induction of diabetes with intravenous STZ at 70-80 mg/kg, it generally takes 6-8 wks for tactile allodynia, the defining symptom of PDN, to fully develop in both rat hindpaws.

Induction of STZ-Diabetes in Rats. On day 0, rats were anaesthetized with 3% isoflurane:97% O₂ and a short polyethylene cannula (0.5 mm ID, 0.8 mm OD) was inserted into the jugular vein to facilitate the intravenous (i.v.) administration of a single dose of streptozotocin (STZ: 70-80 mg/kg). After STZ injection, the cannula was removed and the jugular vein was tied off and the wound was closed. Rats received subcutaneous benzylpenicillin (60 mg) prior to surgery to prevent infection and were kept warm during surgical recovery. Rats were housed singly prior to further experimentation and were monitored daily from the time of STZ administration with regard to general health and well-being.

Water Intake and Body Weight Assessment. Water intake was assessed once-daily on days 7 to 10 post-STZ administration and rats that did not drink 100 mL of water by day 10 were classified as non-diabetic and removed from the study. Body weights were assessed once-weekly.

Measurement of Blood Glucose Levels. Blood glucose levels (BGLs) were assessed using a MEDISENSE® device prior to diabetes induction and on day 10 following STZ administration in those rats whose day 10 water intake was 100 mL. If day 10 BGLs were 15 mM, the diabetes diagnosis was confirmed and the animals remained in the study.

### Pharmacodynamic Assessment

Von Frey Assessment of Paw Withdrawal Thresholds. Calibrated von Frey filaments were used to determine the lowest mechanical threshold required to evoke a brisk paw withdrawal reflex in the rat hindpaws. Briefly, rats were transferred individually to wire mesh testing cages (20 cm×20 cm×20 cm) and allowed to acclimatize for approximately 10-20 min prior to von Frey testing. Commencing with the von Frey filament that produced the 6 g force, the filament was applied to the plantar surface of the hindpaw until the filament buckled slightly. Absence of a response after 3 s prompted use of the next filament of increasing weight. A withdrawal response within 3 s prompted use of the next filament of decreasing weight. Filaments used produced buckling weights of 2, 4, 6, 8, 10, 12, 14, 16, 18 and 20 g. A score of 20 g was given to animals that did not respond to any of the von Frey filaments. Tactile allodynia is regarded as being fully developed when von Frey PWTs in the injured hindpaws are <6 g.

Restoring von-Frey PWTs in the hindpaws from <6 g to pre-STZ surgery levels (12±2 g) is the treatment goal representing full reversal of tactile allodynia. If drug treatment increases PWTs above pre-surgical baseline values (i.e. >12±2 g), such a response is classified as antinociception. Before administration of STZ, baseline von Frey measurements were assessed. Following STZ administration, baseline PWTs were assessed in the hindpaws at week 8-11 to confirm the development of diabetes-induced tactile allodynia.

### Example 1

### Investigation of the Anti-Allodynic Efficacy and Potency of Oral Bolus Dosing Regimens of Tranilast in a Rat Model of Painful Diabetic Neuropathy (PDN): Dose-Response Curve

Preliminary Dose Identification in STZ-Diabetic Rats. Once tactile allodynia was fully developed (PWTs 6 g; 8-11 weeks post-STZ administration), bolus oral doses of vehicle or tranilast (30, 100, 200, 300 mg/kg) or bolus s.c. doses of gabapentin (100 mg/kg) were administered to groups of STZ-diabetic rats. Each rat initially received seven consecutive bolus doses of the same dose magnitude administered twice-daily at 10 to 14 h intervals. Baseline PWTs were assessed prior to the administration of the first, third, fifth and seventh dose of tranilast or gabapentin. Following administration of the seventh oral bolus dose of tranilast, hindpaw PWTs were quantified at the following times: pre-dose, 0.25, 0.5, 0.75, 1, 1.25, 1.5, 2 and 3 h post-dosing. PWTs were determined utilizing the procedure described above. After a 2-3 day “washout” period, rats then received seven consecutive doses of another oral dosing regimen of tranilast or of s.c. gabapentin (100 mg/kg) administered twice-daily at 10-14 h intervals, with each animal receiving up to 5 dosing regimens. To produce a dose-response curve, STZ-diabetic rats received either tranilast at 30, 100, 200 or 300 mg/kg and the anti-allodynic responses were compared with those produced by s.c. gabapentin (100 mg/kg) as the positive control or vehicle.

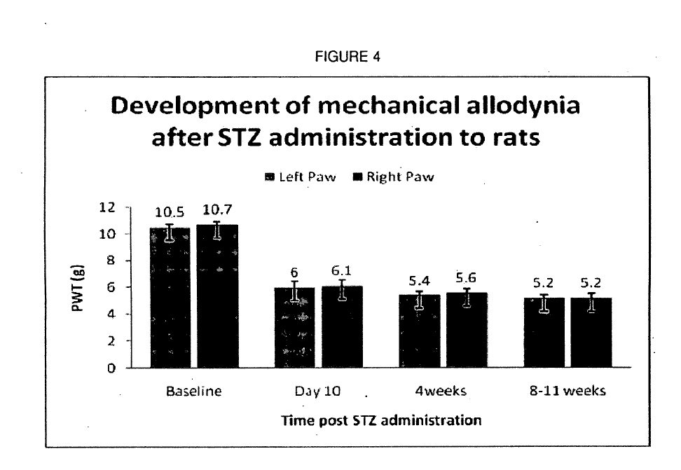

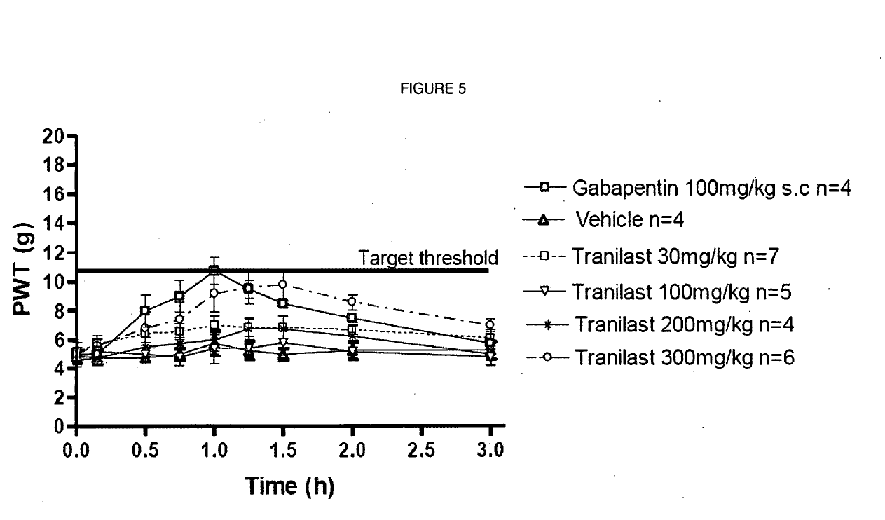

Following administration of 7 consecutive s.c. bolus doses of gabapentin (100 mg/kg) according to a twice-daily dosing regimen in STZ-diabetic rats administered multiple dosing regimens, there was significant relief of tactile (mechanical) allodynia in the hindpaws (FIG. 4). Peak anti-allodynia in the hindpaws occurred between 0.75-1.25 h post-dosing and the corresponding duration of action was >3 h. Specifically, the mean (±SEM) PWT increased from 4.5 (±0.5) g pre-dose to 11.0 (±1.0)_{g} at the mean time of peak response (1 h post-dosing). Following administration of 7 consecutive oral bolus doses of tranilast according to a twice-daily regimen in STZ-diabetic rats administered multiple dosing regimens, significant anti-allodynia was produced only by the 300 mg/kg dosing regimen (FIG. 5). Peak anti-allodynia in the hindpaws occurred between 1-1.5 h post-dosing and the corresponding duration of action was >3 h. Specifically, the mean (±SEM) PWT increased from 5.1 (±0.4)_{g} pre-dose to 9.8 (±1.1)_{g} at the mean time of peak response (1.5 h post-dosing).

### Example 2

### Investigation of the Anti-Allodynic Efficacy and Potency of Oral Bolus Dosing Regimens of Tranilast in Drug Naive STZ-Diabetic Rats

Dose-Response Curve for Tranilast in Drug-Naive STZ-Diabetic Rats. Once tactile allodynia was fully developed (PWTs 6 g; 8-11 weeks post-STZ administration), oral bolus doses of tranilast at 100 (n=4), 200 (n=4), 300 (n=5) or 400 (n=4) mg/kg were administered to drug-naive STZ-diabetic rats for 7-consecutive doses. Control animals received s.c. bolus doses of gabapentin (100 mg/kg; n=4) or oral bolus doses of vehicle (n=4), using the same dosing regimen outlined above. PWTs were determined utilizing the procedure outlined above. Baseline PWTs were measured prior to the administration of the first, third, fifth and seventh dose of tranilast, gabapentin or vehicle. Animals also underwent intensive von Frey testing (0.25, 0.5, 0.75, 1, 1.25, 1.5, 2 and 3 h post-dosing) after administration of the seventh consecutive bolus dose administered twice-daily at 10-14 h intervals.

Behavioural Observations. STZ-diabetic rats were monitored for visible and audible signs of distress throughout the testing period. The visible signs of distress included behavioural changes such as complete immobility, movement with abnormal gait, agitation, aggression, wet dog shakes, excessive grooming, restlessness with constant movement, repeated sudden movements or staring.

Rat Euthanasia and Disposal. After completion of the experimental protocol, rats were euthanised with 100% CO₂ followed by cervical dislocation. Rat carcasses were frozen until removal by The University of Queensland biological waste removal service.

Data Analysis. Mean (±SEM) PWT versus time curves were plotted for the 7ₜₕ oral bolus dose of each dosing regimen of tranilast, gabapentin or vehicle in STZ-diabetic rats. Percent maximal possible reversal

$$\% MPR = \frac{Post \text{-} dose PWT ( g ) - Pre \text{-} dose PWT ( ( g )}{Pre \text{-} injury PWT ( g ) - Pre \text{-} dose PWT ( g )} \times \frac{100}{1}$$

PWT values were also normalized by subtracting the respective pre-dosing baseline values and the areas under the normalized response versus time curves (AUC values) were estimated using trapezoidal integration.

Statistical Analysis. The Mann-Whitney test was used to compare differences in the values between treatment groups. Statistical analysis was undertaken using the GraphPad Prism™ software package v3.0, and the statistical significance criterion was P<0.05.

### Results

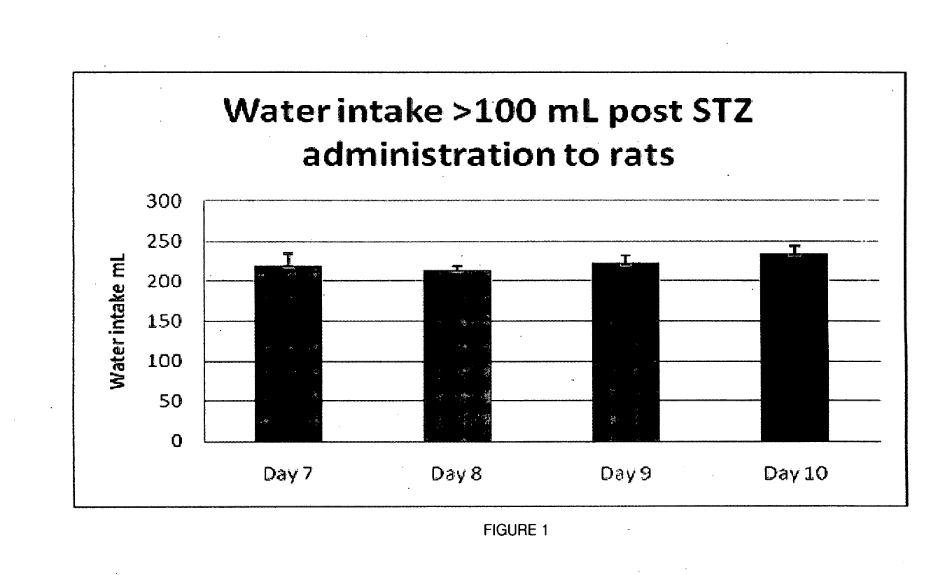

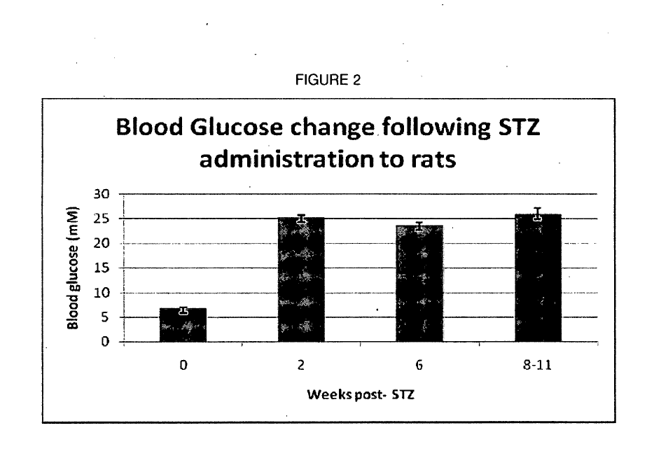

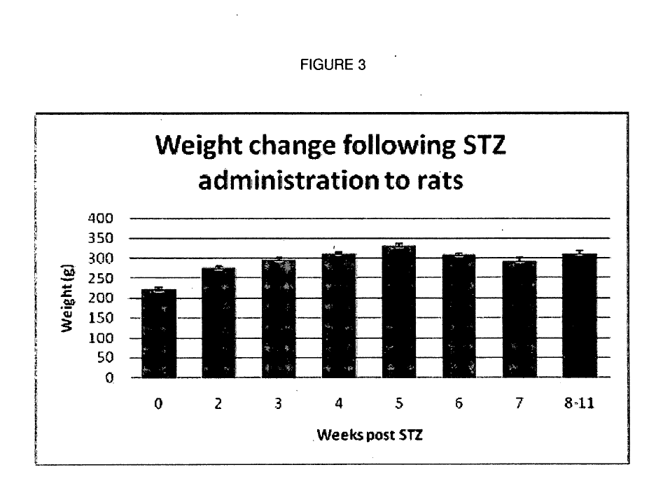

Induction of PDN. The data were determined in STZ-treated rats classified as “diabetic” such that water intake was 100 mL per day by day 10 post-STZ administration (FIG. 1) and blood glucose levels were 15 mM at 10 days, 2 and 8-11 weeks post-STZ administration (FIG. 2). Weight was assessed once-weekly until administration of compounds (FIG. 3). “8-11 weeks” is the data obtained immediately prior to administration of the first dose of compound, which occurred from 8-11 weeks post-STZ administration depending on the group of rats tested. Once developed, mechanical allodynia remains very stable for up to 24 weeks post-STZ.

PDN-Tactile Allodynia (von Frey Paw Withdrawal Thresholds). Following the induction of diabetes with i.v. STZ in rats, tactile (mechanical) allodynia developed progressively over 6-8 weeks in both hindpaws, consistent with expectations. The time of initiation of treatment with the test article or vehicle or gabapentin was 8-11 weeks post-STZ administration, i.e. after tactile allodynia was well developed. Specifically, the mean (SEM) von Frey PWT values for the left hindpaw decreased significantly (p<0.05) from 10.5 (±0.2) g to 5.2 (±0.2) g by 8-11 weeks post-STZ administration and from 10.7 (±0.2) g to 5.2 (±0.3) g for the right hindpaw (FIG. 4).

As expected, the administration of STZ affected the left and right hindpaws to a similar extent as there were no significant differences in the mean (SEM) PWT values between the left (5.2 0.2 g) and right (5.2 0.3 g) hindpaws. For this reason, mean data for both hindpaws has been included in the remainder of this report and the mean pre-STZ value is designated as the target threshold.

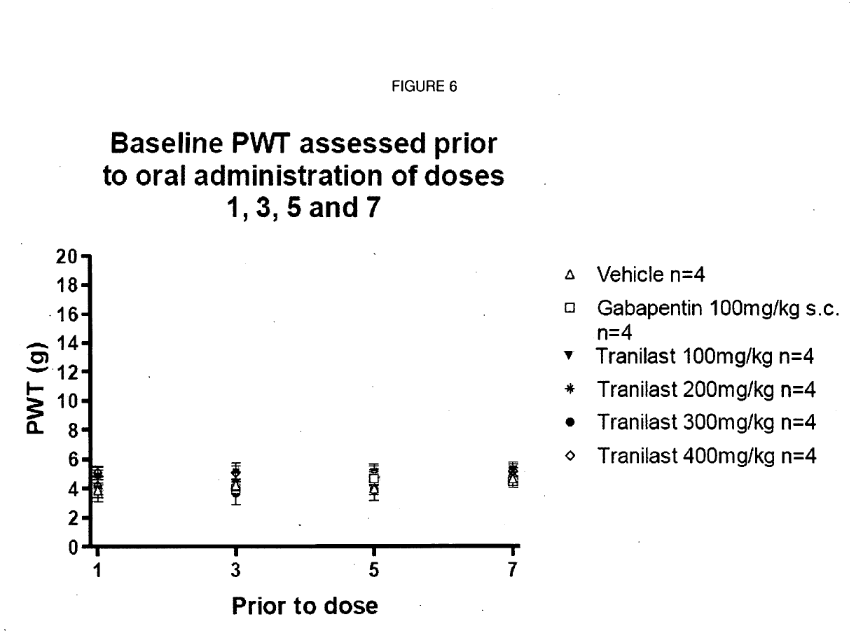

Baseline PWT thresholds were not significantly different following oral bolus doses of vehicle, gabapentin, or tranilast, at all doses, when assessed prior to dose 1, 3, 5 and 7 (FIG. 6, Table 1).

TABLE 1
Mean (±SEM) von Frey Paw Withdrawal thresholds assessed prior
to doses 1, 3, 5 and 7 to drug-naïve STZ-diabetic rats

| Time | | Gabapentin | Tranilast | Tranilast | Tranilast | Tranilast |
|---|---|---|---|---|---|---|
| (h) | Vehicle | 100 mg/kg s.c. | 100 mg/kg | 200 mg/kg | 300 mg/kg | 400 mg/kg |
| Prior to | 3.9 ± 0.5 | 3.7 ± 0.6 | 4.8 ± 0.5 | 4.9 ± 0.6 | 4.2 ± 0.6 | 5.1 ± 0.5 |
| dose 1 | | | | | | |
| Prior to | 4.25 ± 0.4 | 4.0 ± 0 | 4.4 ± 0.7 | 5.2 ± 0.6 | 3.7 ± 0.8 | 5.1 ± 0.5 |
| dose 3 | | | | | | |
| Prior to | 4.0 ± 0.4 | 4.7 ± 0.5 | 5.1 ± 0.5 | 5.2 ± 0.5 | 3.9 ± 0.7 | 4.8 ± 0.5 |
| dose 5 | | | | | | |
| Prior to | 4.7 ± 0.5 | 4.5 ± 0.4 | 5.3 ± 0.5 | 5.2 ± 0.5 | 4.8 ± 0.6 | 5.2 ± 0.5 |
| dose 7 | | | | | | |

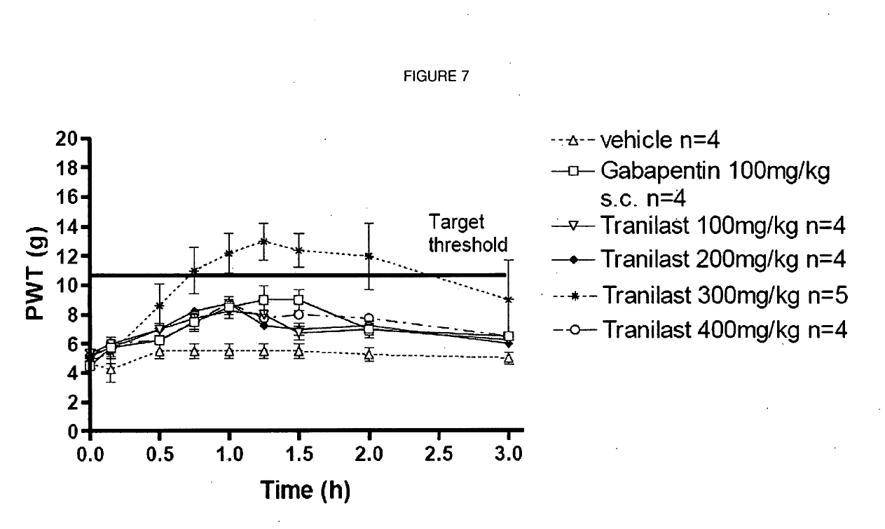

Following s.c. administration of 7 consecutive bolus doses of gabapentin (100 mg/kg) according to a twice-daily dosing regimen in drug-naïve STZ-diabetic rats, there was significant relief of tactile allodynia produced in the rat hindpaws (FIG. 7; Table 2). Peak anti-allodynia in the hindpaws occurred between 1-1.5 h post-dosing and the corresponding mean duration of action was >3 h. Specifically, the mean (±SEM) PWT increased from 4.5 (±0.2) g pre-dose to 9.0 (±1.0) g at the time of peak response (1.25 h post-dosing). Consistent with expectations, bolus doses of vehicle did not produce significant anti-allodynia.

TABLE 2
Mean (±SEM) von Frey Paw Withdrawal thresholds assessed following administration
of the 7th consecutive dose to drug-naïve STZ-diabetic rats.

| Time | | Gabapentin | Tranilast | Tranilast | Tranilast | Tranilast |
|---|---|---|---|---|---|---|
| (h) | Vehicle | 100 mg/kg s.c. | 100 mg/kg | 200 mg/kg | 300 mg/kg | 400 mg/kg |
| 0 | 4.67 ± 0.4 | 4.5 ± 0.2 | 5.3 ± 0.4 | 5.2 ± 0.4 | 4.8 ± 0.5 | 5.2 ± 0.4 |
| 0.15 | 4.25 ± 0.8 | 5.75 ± 0.2 | 6.0 ± 0 | 5.75 ± 0.7 | 5.4 ± 0.7 | 6.0 ± 0 |
| 0.5 | 5.5 ± 0.5 | 6.25 ± 0.2 | 7.25 ± 0.5 | 7.25 ± 0.5 | 8.6 ± 1.5 | 6.25 ± 0.2 |
| 0.75 | 5.5 ± 0.5 | 7.5 ± 0.6 | 7.75 ± 0.25 | 8.25 ± 0.25 | 11 ± 1.6 | 7.5 ± 0.5 |
| 1 | 5.5 ± 0.5 | 8.5 ± 0.5 | 8.25 ± 0.5 | 8.75 ± 0.5 | 12.2 ± 1.3 | 8.75 ± 0.25 |
| 1.25 | 5.5 ± 0.5 | 9.0 ± 1.0 | 8.0 ± 0 | 7.25 ± 0.25 | 13 ± 1.3 | 7.8 ± 0.25 |
| 1.5 | 5.5 ± 0.5 | 9.0 ± 0.7 | 6.75 ± 0.5 | 7.0 ± 0.5 | 12.4 ± 1.1 | 8.0 ± 0 |
| 2 | 5.25 ± 0.5 | 7.0 ± 0.4 | 7.0 ± 0.6 | 7.25 ± 0.25 | 12.0 ± 2.2 | 7.8 ± 0.25 |
| 3 | 5.0 ± 0.4 | 6.5 ± 0.3 | 6.25 ± 0.3 | 6.0 ± 0 | 9.0 ± 2.7 | 6.5 ± 0.3 |

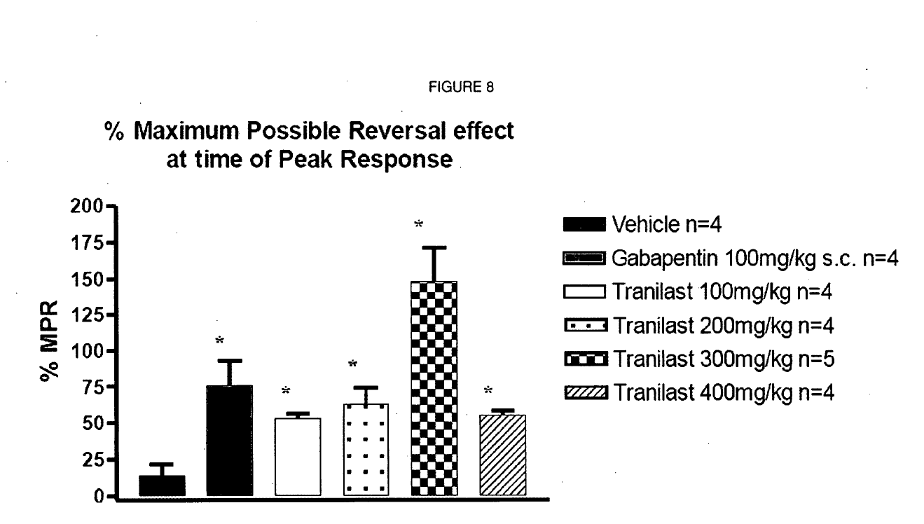

Following twice-daily administration of 7 consecutive oral bolus doses of tranilast in STZ-diabetic rats, at 100, 200, 300 and 400 mg/kg, there was significant relief of tactile allodynia in the rat hindpaws (FIG. 8). Specifically, the peak anti-allodynic response following twice-daily administration of 7 consecutive oral bolus doses of tranilast in STZ-diabetic rats, at 100, 200, 300 and 400 mg/kg was significantly (p<0.05) greater than the response following twice-daily administration of 7 consecutive oral bolus doses of vehicle in STZ-diabetic rats. The maximum anti-allodynic effect appeared to be produced by the 300 mg/kg dosing regimen of tranilast as the anti-allodynic response produced by the 400 mg/kg dosing regimen was lower than that for the 300 mg/kg dosing regimen.

Following administration of 7 consecutive twice-daily oral bolus doses of tranilast at 300 mg/kg in drug-naïve STZ-diabetic rats (n=5), there was significant relief of tactile allodynia in the hindpaws (FIG. 5). Peak anti-allodynia occurred between 1-1.5 h post-dosing and the corresponding mean duration of action was >3 h. Specifically, the mean (±SEM) PWT increased from 4.8 (±0.5)_{g} pre-dose to 13.0 (±1.3)_{g} at the time of peak response (1.25 h post-dosing). At the highest dose tested (400 mg/kg), peak anti-allodynia in the hindpaws of STZ-diabetic rats occurred between 0.75-1.25 h post-dosing and the corresponding mean duration of action was 3 h. Specifically, the mean (±SEM) PWT increased from 5.2 (±0.4)_{g} pre-dose to 8.8 (±0.3)_{g} at the time of peak response (1 h post-dosing). Twice-daily oral administration of 7 consecutive doses of vehicle did not produce significant anti-allodynia, indicating that neither the vehicle nor the testing procedure significantly altered PWT values in the hindpaws of STZ-diabetic rats.

The above data was converted to % maximum pain relief (MPR), with 100% MPR representing the full reversal of tactile allodynia in the hindpaws of drug-naïve rats following STZ-administration. FIG. 8 clearly demonstrates that following administration of the 7ₜₕ consecutive bolus dose, administered according to a twice-daily dosing regimen, the % MPR at the time of peak effect for gabapentin (s.c. 100 mg/kg) and tranilast at 100, 200, 300 and 400 mg/kg are significantly greater than that for vehicle (Table 3).

TABLE 3
Mean (±SEM) peak responses and AUC values
for drug-naïve STZ-diabetic rats.

| Peak response | % MPR at peak | | AUC |
|---|---|---|---|
| Vehicle | 5.5 ± 0.5 | 13.8 ± 8.1 | 2.3 ± 1.2 |
| Gabapentin 100 mg/kg s.c. | 9.0 ± 1.0 | 75.8 ± 17.2 | 8.3 ± 0.4 |
| Tranilast 100 mg/kg | 8.25 ± 0.5 | 53.2 ± 3.3 | 4.98 ± 1.7 |
| Tranilast 200 mg/kg | 8.75 ± 0.5 | 62.6 ± 11.7 | 5.75 ± 0.7 |
| Tranilast 300 mg/kg | 13.0 ± 1.3 | 147.8 ± 23.6 | 17.2 ± 3.3 |
| Tranilast 400 mg/kg | 8.75 ± 0.25 | 62.2 ± 4.2 | 6.3 ± 0.9 |

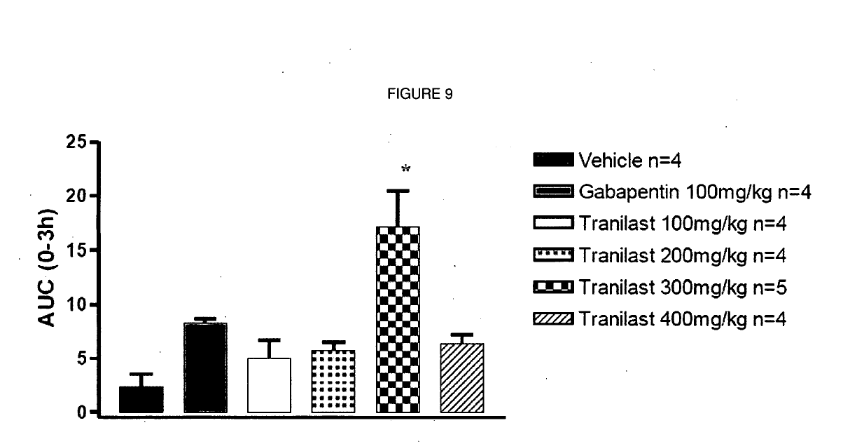

FIG. 9 shows that the mean (±SEM) area under the anti-allodynic response versus time curve (AUC) in the ipsilateral hindpaw following administration of the 7ₜₕ consecutive oral bolus dose of tranilast at 300 mg/kg is significantly greater (p<0.05) than that of vehicle, in drug-naïve STZ-diabetic rats. Although the AUC values for the 100, 200 and 400 mg/kg doses were larger than those for vehicle, these did not reach statistical significance, most likely due to the small “n” numbers.

Adverse effects. For STZ-diabetic rats that received 7 consecutive bolus doses of oral tranilast (30-400 mg/kg) or s.c. gabapentin (100 mg/kg), there were no discernible adverse effects observed at the doses assessed.

Following twice-daily administration of 7 consecutive s.c. bolus doses of gabapentin at 100 mg/kg in STZ-diabetic rats, there was significant relief of tactile allodynia in the hindpaws, as expected. Twice-daily administration of 7 consecutive oral bolus doses of tranilast at 100-300 mg/kg produced dose-dependent relief of tactile allodynia in STZ-diabetic rats, when each rat received up to 5 different dosing regimens, with each dosing regimen separated by a 2-3 day “washout” period. The peak anti-allodynic response produced by the 7ₜₕ consecutive 300 mg/kg oral dose of tranilast was similar to that produced by the corresponding dose of s.c. gabapentin at 100 mg/kg, when administered to STZ-diabetic rats according to a “washout” protocol.

Baseline PWT thresholds were not significantly different following oral bolus doses of vehicle, gabapentin, or tranilast (at all doses) when assessed prior to doses 1, 3, 5 and 7 to drug-naïve STZ-diabetic rats, consistent with the notion that the anti-allodynic effect of the compound was related to its pharmacokinetics and that the disease state was not altered by the administration of 7 consecutive doses of these compounds at twice-daily intervals.

Following twice-daily administration of 7 consecutive s.c. bolus doses of gabapentin at 100 mg/kg to drug-naïve STZ-diabetic rats, there was significant relief of tactile allodynia in the rat hindpaws. Similarly, following twice-daily administration of 7 consecutive oral bolus doses of tranilast at 100, 200, 300 and 400 mg/kg in drug-naïve STZ-diabetic rats, anti-allodynia was produced in the rat hindpaws. The peak anti-allodynic response and % MPR following twice-daily administration of 7 consecutive oral bolus doses of tranilast at 100, 200, 300 and 400 mg/kg was significantly greater than the response following twice-daily administration of 7 consecutive oral bolus doses of vehicle in drug-naïve STZ-diabetic rats. The area under the anti-allodynia versus time curve (AUC) following twice-daily administration of 7 consecutive oral bolus doses of tranilast at 300 mg/kg was significantly greater than the response following twice-daily administration of 7 consecutive oral bolus doses of vehicle in drug-naïve STZ-diabetic rats.

Importantly, twice-daily administration of 7 consecutive bolus doses of s.c. gabapentin (100 mg/kg) or oral tranilast (30-400 mg/kg) did not produce discernible adverse behavioural effects in STZ-diabetic rats, at the doses assessed.

1. A method of decreasing neuropathic pain in a mammal, said method comprising:
administering to said mammal an effective amount of a compound of formula I: 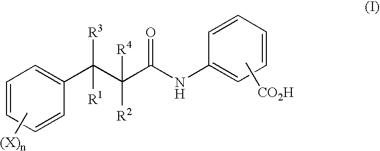
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alkyl or alkoxy groups, they may be connected together to form a ring, and n is an integer from 1 to 3
for a period sufficient to decrease pain.

2. The method of claim 1, wherein the compound is chosen from 2-[[3-(2-methylphenyl)-1-oxo-2-propenyl]amino]benzoic acid; 2-[[3-(3-methylphenyl)-1-oxo-2-propenyl]amino]benzoic acid; 2-[[3-(4-methylphenyl)-1-oxo-2-propenyl]amino]benzoic acid; 2-[[3-(2-ethylphenyl)-1-oxo-2-propenyl]amino]benzoic acid; 2-[[3-(3-ethylphenyl)-1-oxo-2-propenyl]amino]benzoic acid; 2-[[3-(4-ethylphenyl)-1-oxo-2-propenyl]amino]benzoic acid; 2-[[3-(2-propylphenyl)-1-oxo-2-propenyl]amino]benzoic acid; 2-[[3-(3-propylphenyl)-1-oxo-2-propenyl]amino]benzoic acid; 2-[[3-(4-propylphenyl)-1-oxo-2-propenyl]amino]benzoic acid; 2-[[3-(2-hydroxyphenyl)-1-oxo-2-propenyl]amino]benzoic acid; 2-[[3-(3-hydroxyphenyl)-1-oxo-2-propenyl]amino]benzoic acid; 2-[[3-(4-hydroxyphenyl)-1-oxo-2-propenyl]amino]benzoic acid; 2-[[3-(2-chlorophenyl)-1-oxo-2-propenyl]amino]benzoic acid; 2-[[3-(3-chlorophenyl)-1-oxo-2-propenyl]amino]benzoic acid; 2-[[3-(4-chlorophenyl)-1-oxo-2-propenyl]amino]benzoic acid; 2-[[3-(2-fluorophenyl)-1-oxo-2-propenyl]amino]benzoic acid; 2-[[3-(3-fluorophenyl)-1-oxo-2-propenyl]amino]benzoic acid; 2-[[3-(4-fluorophenyl)-1-oxo-2-propenyl]amino]benzoic acid; 2-[[3-(2-bromophenyl)-1-oxo-2-propenyl]amino]benzoic acid; 2-[[3-(3-bromophenyl)-1-oxo-2-propenyl]amino]benzoic acid; 2-[[3-(4-bromophenyl)-1-oxo-2-propenyl]amino]benzoic acid; 2-[[3-(2,3-dimethoxyphenyl)-1-oxo-2-propenyl]amino]benzoic acid; 2-[[3-(3,4-dimethoxyphenyl)-1-oxo-2-propenyl]amino]benzoic acid; 2-[[3-(2,4-dimethoxyphenyl)-1-oxo-2-propenyl]amino]benzoic acid; 2-[[3-(2,3-dimethylphenyl)-1-oxo-2-propenyl]amino]benzoic acid; 2-[[3-(3,4-dimethylphenyl)-1-oxo-2-propenyl]amino]benzoic acid; 2-[[3-(2,4-dimethylphenyl)-1-oxo-2-propenyl]amino]benzoic acid; 2-[[3-(2,3-diethoxyphenyl)-1-oxo-2-propenyl]amino]benzoic acid; 2-[[3-(3,4-diethoxyphenyl)-1-oxo-2-propenyl]amino]benzoic acid; 2-[[3-(2,4-diethoxyphenyl)-1-oxo-2-propenyl]amino]benzoic acid; 2-[[3-(2,3-dipropoxyphenyl)-1-oxo-2-propenyl]amino]benzoic acid; 2-[[3-(3,4-dipropoxyphenyl)-1-oxo-2-propenyl]amino]benzoic acid; 2-[[3-(2,4-dipropoxyphenyl)-1-oxo-2-propenyl]amino]benzoic acid; 2-[[3-(2,3-diethylphenyl)-1-oxo-2-propenyl]amino]benzoic acid; 2-[[3-(3,4-diethylphenyl)-1-oxo-2-propenyl]amino]benzoic acid; 2-[[3-(2,4-diethylphenyl)-1-oxo-2-propenyl]amino]benzoic acid; 2-[[3-(2,3-dipropylphenyl)-1-oxo-2-propenyl]amino]benzoic acid; 2-[[3-(3,4-dipropylphenyl)-1-oxo-2-propenyl]amino]benzoic acid; 2-[[3-(2,4-dipropylphenyl)-1-oxo-2-propenyl]amino]benzoic acid; 2-[[3-(2-methoxy-3-methylphenyl)-1-oxo-2-propenyl]amino]benzoic acid; 2-[[3-(3-methoxy-4-methylphenyl)-1-oxo-2-propenyl]amino]benzoic acid; 2-[[3-(2-methoxy-3-methylphenyl)-1-oxo-2-propenyl]amino]benzoic acid; 2-[[3-(2-methoxy-4-methylphenyl)-1-oxo-2-propenyl]amino]benzoic acid; 2-[[3-(2-methoxy-3-chlorophenyl)-1-oxo-2-propenyl]amino]benzoic acid; 2-[[3-(3-methoxy-4-chlorophenyl)-1-oxo-2-propenyl]amino]benzoic acid; 2-[[3-(2-methoxy-3-chlorophenyl)-1-oxo-2-propenyl]amino]benzoic acid; 2-[[3-(2-methoxy-4-chlorophenyl)-1-oxo-2-propenyl]amino]benzoic acid; 2-[[3-(2-methoxy-3-hydroxyphenyl)-1-oxo-2-propenyl]amino]benzoic acid; 2-[[3-(3-methoxy-4-hydroxyphenyl)-1-oxo-2-propenyl]amino]benzoic acid; 2-[[3-(2-methoxy-3-hydroxyphenyl)-1-oxo-2-propenyl]amino]benzoic acid; 2-[[3-(2-methoxy-4-hydroxyphenyl)-1-oxo-2-propenyl]amino]benzoic acid; 2-[[3-(3,4-trimethylenephenyl)-1-oxo-2-propenyl]amino]benzoic acid; 2-[[3-(2,3-trimethylenephenyl)-1-oxo-2-propenyl]amino]benzoic acid; 2-[[3-(3,4-methylenedioxyphenyl)-1-oxo-2-propenyl]amino]benzoic acid; and 2-[[3-(3,4-ethylenedioxyphenyl)-1-oxo-2-propenyl]amino]benzoic acid.

3. The method of any one of claims 1, wherein the compound is 2-[[3-(3,4-dimethoxyphenyl)-1-oxo-2-propenyl]amino]benzoic acid (tranilast, TNL) or a pharmacologically acceptable salt thereof.

4. The method of claim 3, wherein the tranilast is orally administered.

5. The method of claim 4, wherein the neuropathic pain is associated with painful diabetic neuropathy.

6. The method of claim 5, wherein tranilast is administered at least once daily for at least 3 days.

7. The method of claim 6, wherein tranilast is administered at least twice daily.

8. The method of claim 6 wherein tranilast is administered for at least one week.

9. The method according to claim 1, further comprising administering a second agent effective in the treatment of neuropathic pain.

10. A composition for use in any of the methods of claim 1.

11. The use of a compound of formula I for the manufacture of a medicament for a method according to any one of claim 1.
